(19) United States
(12) Patent Application Publication
(10) Pub. No.: US 2006/0052786 A1
(43) Pub. Date: Mar. 9, 2006

(54) Polyaxial device for spine stabilization during osteosynthesis

(75) Inventors: Jack A. Dant, St. Paul, MN (US); Paul F. Boschert, Minneapolis, MN (US); Angela L. Hillyard, Greenfield, MN (US)

Correspondence Address: FAEGRE & BENSON;ATTN: PATENT DOCKETING, 2200 WELLS FARGO CENTER, 90 SOUTH 7TH STREET, MINNEAPOLIS, MN, 55402-3901, US

(73) Assignee: Zimmer Spine, Inc., Minneapolis, MN (US)

(21) Appl. No.: 11/247,715

(22) Filed: Oct. 11, 2005

Related U.S. Application Data
(63) Continuation-in-part of application No. 11/195,838, filed on Aug. 3, 2005.
Application No. 11/195,838 is a continuation-in-part of application No. 10/920,729, filed on Aug. 17, 2004.
(60) Provisional application No. 60/617,882, filed on Oct. 11, 2004.

(51) Int. Cl. A61B 17/70

(52) U.S. Cl. 606/61

(57) ABSTRACT

An orthopedic fixation device and method for correction and fixation of the vertebrae to facilitate an anatomically correct fusion is provided. The orthopedic fixation device includes an elongated plate including at least one fastener opening, at least two cup shaped washers mounted to the plate, and an anchor mounted to each washer, wherein each cup shaped washer slides on a track that extends along each fastener opening and wherein each cup shaped washer includes a top flange that is captured within the track that allows it to slide.

This application is a continuation-in-part of the U.S. patent application Ser. No. 11/195,838, filed Aug. 3, 2005, which is a continuation-in-part of the U.S. patent application Ser. No. 10/920,729, filed Aug. 17, 2004. This application also claims the benefit of the Provisional Application Ser. No. 60/617,882, filed Oct. 11, 2004. The Provisional Application is incorporated herein by reference.

### TECHNICAL FIELD

The principles disclosed herein relate generally to bone fixation and stabilization devices. More specifically, the disclosure relates to intervertebral connection systems suited for stabilization of the spine.

### BACKGROUND

The spinal column is a highly complex system of bones and connective tissues that provides support for the body and protects the delicate spinal cord and nerves. The spinal column includes a series of vertebrae stacked one atop the other, each vertebral body including an inner or central portion of relatively weak cancellous bone and an outer portion of relatively strong cortical bone. Situated between each vertebral body is an intervertebral disc that cushions and dampens compressive forces experienced by the spinal column. A vertebral canal containing the spinal cord and nerves is located behind the vertebral bodies.

There are many types of spinal column disorders including scoliosis (abnormal lateral curvature of the spine), kyphosis (abnormal forward curvature of the spine, usually in the thoracic spine), excess lordosis (abnormal backward curvature of the spine, usually in the lumbar spine), spondylolisthesis (forward displacement of one vertebra over another, usually in a lumbar or cervical spine) and other disorders caused by abnormalities, disease or trauma, such as ruptured or slipped discs, degenerative disc disease, fractured vertebra, and the like. Patients that suffer from such conditions usually experience extreme and debilitating pain, as well as diminished nerve function.

The present invention generally involves a technique commonly referred to as spinal fixation whereby surgical implants are used for fusing together and/or mechanically immobilizing vertebrae of the spine. Spinal fixation may also be used to alter the alignment of adjacent vertebrae relative to one another so as to change the overall alignment of the spine. Such techniques have been used effectively to treat the above-described conditions and, in most cases, to relieve pain suffered by the patient. However, as will be set forth in more detail below, there are some disadvantages associated with current fixation devices.

One spinal fixation technique involves immobilizing the spine by using orthopedic rods, commonly referred to as spinal rods, that nun generally parallel to the spine. This may be accomplished by exposing the spine posteriorly and fastening bone screws to the pedicles of the appropriate vertebrae. Clamping elements adapted for receiving a spinal rod therethrough are then used to join the spinal rods to the screws. The aligning influence of the rods forces the spine to conform to a more desirable shape. In certain instances, the spinal rods may be bent to achieve the desired adjustment of the spinal column. Some examples of such spinal stabilization systems are disclosed in U.S. Pat. Nos. 6,074,391; 6,488,681; 6,280,442; 5,879,350; 6,371,957 B1; 6,355,040; 6,050,997; 5,882,350; 6,248,105; 5,443,467; 6,113,601; 5,129,388; 5,733,286; 5,672,176; and 5,476,464, the entire disclosures of which are incorporated herein by reference.

U.S. Pat. No. 5,129,388 to Vignaud et al. discloses a spinal fixation device including a pedicle screw having a U-shaped head rigidly connected to an upper end of the screw. The U-shaped head includes two arms forming a U-shaped channel for receiving a spinal rod therein. The U-shaped head is internally threaded so that a setscrew having external threads may be screwed therein. After the pedicle screw has been inserted into bone and a spinal rod positioned in the U-shaped channel, the setscrew is threaded into the internal threads of the U-shaped channel for securing the spinal rod in the channel and resisting relative movement between the spinal rod and the pedicle screw.

Surgeons have encountered considerable difficulty when attempting to implant spinal fixation devices such as those disclosed in the above-mentioned '388 patent. This is because the U-shaped heads of adjacent screws are often out of alignment with one another due to spine curvature and the different orientations of the pedicles receiving the screws. As a result, spinal rods must often be bent in multiple planes in order to pass the rods through adjacent U-shaped channels. These problems weaken the strength of the assembly and result in significantly longer operations, thereby increasing the likelihood of complications associated with surgery.

In response to the above-noted problems, U.S. Pat. No. 5,733,286 to Errico et al, U.S. Pat. No. 5,672,176 to Biedermann et al., and U.S. Pat. No. 5,476,464 to Metz-Stavenhagen disclose polyaxial spinal fixation devices wherein the anchoring element fixed to the bone has a spherically-shaped head. The fixation devices in the above-identified patents also have orthopedic rod capturing assemblies for securing orthopedic rods in the capturing assemblies and connecting the rods with the anchoring elements. The spherically shaped heads of the anchoring elements permit movement of the anchoring elements relative to the orthopedic rod capturing assemblies.

There remains room for improvement of prior art spinal fixation devices. What are needed in the art are devices allowing for axial fixation between the adjacent vertebrae without the sizing, bending and cutting associated with conventional rod and saddle constructs. Also needed are devices that include fewer separate components for facilitating manipulation of the relative parts during all operative phases to reduce surgical time. What are also needed are fixation devices that provide enhanced stability with smaller overall profiles than conventional connector-rod constructs.

### SUMMARY

One inventive aspect of the disclosure relates to polyaxial anchor type orthopedic fixation devices adapted to simplify the surgical procedures required to provide stabilization between vertebral bodies.

It should be noted that, at various locations throughout the specification, guidance is provided through lists of examples. The examples are for illustrative purposes and are not intended to limit the scope of the invention.

### BRIEF DESCRIPTION OF THE DRAWINGS

FIG. 1 is an exploded perspective view of an orthopedic fixation device having features that are examples of inventive aspects disclosed herein;

FIG. 2 is a cross-sectional view of the embodiment of the orthopedic fixation device of FIG. 1 taken along a vertical cross-sectional plane that bisects the device;

FIG. 3 is a top view of the embodiment of the orthopedic fixation device of FIG. 1, showing the device mounted on the spine from a posterior approach;

FIG. 4 is a top view of an embodiment of the invention in which the two orthopedic fixation devices of FIG. 3 are linked to each other by a transverse connector;

FIG. 5 is a top view of a plate of the orthopedic fixation device of FIG. 1;

FIG. 6 is a bottom view of the plate of the orthopedic fixation device of FIG. 1;

FIG. 7 is a cross-sectional view of the plate of the orthopedic fixation device of FIG. 1 taken along line 7-7 of FIG. 5;

FIG. 8 is a side view of another embodiment of a plate having features that are examples of inventive aspects disclosed herein, the plate has a bent bridge portion;

FIG. 9 is a top view of still another embodiment of a plate having features that are examples of inventive aspects disclosed herein, the plate has three fastener openings;

FIG. 10 is a top view of a cup-shaped washer of the orthopedic fixation device of FIG. 1;

FIG. 11 is a cross-sectional view of the cup-shaped washer of the orthopedic fixation device of FIG. 1 taken along line 11-11 of FIG. 10;

FIG. 12 is a partial side view of still another embodiment of a plate having features that are examples of inventive aspects disclosed herein, the plate including an integral, non-sliding cup-shaped washer, illustrated with hidden lines;

FIG. 13 is a perspective view of still another embodiment of a plate having features that are examples of inventive aspects disclosed herein, the plate has a stepped bridge portion, the plate is illustrated coupled to the washers of FIGS. 1-4 and 10-11;

FIG. 14 is a perspective view of a bone anchor and a toggle bolt of the orthopedic fixation device of FIG. 1;

FIG. 15 is a perspective view of a transverse connector having features that are examples of inventive aspects disclosed herein;

FIG. 16 is a side view of the transverse connector of FIG. 15; and

FIG. 17 is an alternate embodiment of the present invention showing two transversely connected orthopedic fixation devices mounted on a lateral side of the spine.

### DETAILED DESCRIPTION

The inventive aspects of the disclosure will now be described by reference to the several drawing figures. The functional features of the invention can be embodied in any number of specific configurations. It will be appreciated, however, that the illustrated embodiments are provided for descriptive purposes and should not be used to limit the invention. Although the disclosure will be described in terms of spinal fixation, the fixation device can be utilized in any type of orthopedic fixation.

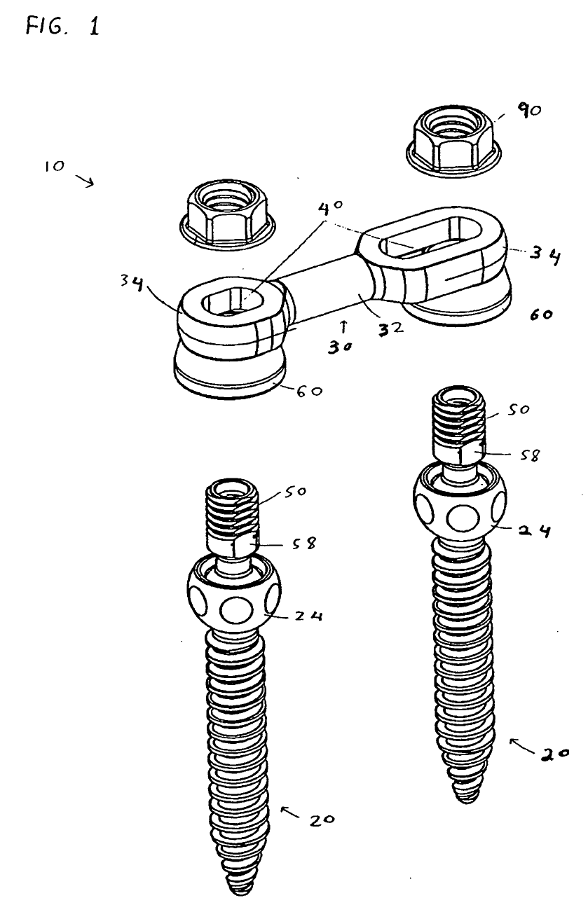

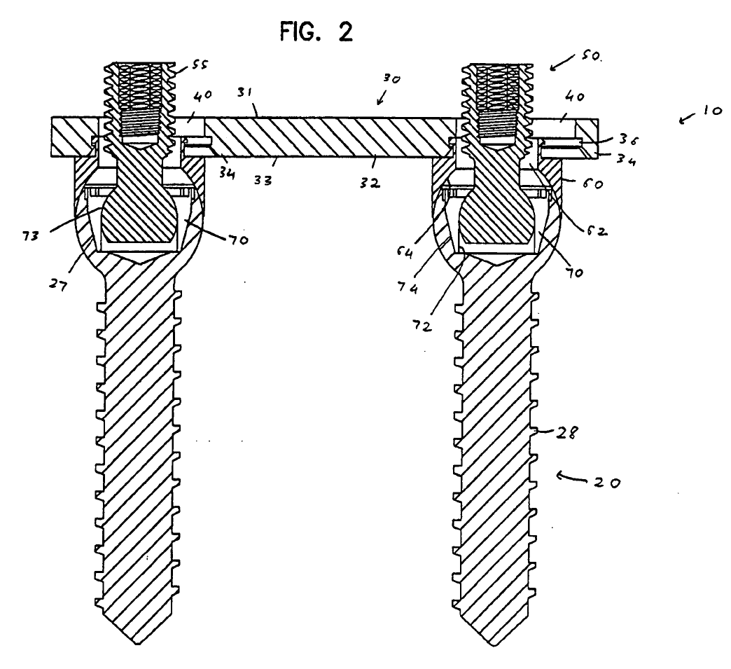

FIGS. 1 and 2 illustrate one embodiment of an orthopedic fixation device 10 having features that are examples of inventive aspects in accordance with the principles of the present disclosure. The fixation device 10 includes a plate 30 having fastener openings 40 for receiving portions of bone anchors 20. The fastener openings 40 can allow for linear slidability and adjustment of bone anchors 20 relative to the plate 30. The fixation device 10 also includes cup-shaped washers 60 that are slidably mounted to the plate openings 40 between the bone anchors 20 and the plate 30. Each bone anchor 20 of the fixation device includes a generally spherical head 24 including an interior cavity that forms a ball/socket coupling arrangement with a toggle bolt 50. The ball/socket arrangement allows for polyaxial movement of the bone anchor 20 relative to the toggle bolt 50. The toggle bolts 50 are received through the fastener opening(s) 40 of the plate 30 as the spherical heads 24 of the bone anchors 20 fit within the cup-shaped washers 60. The fixation device further includes a nut 90 (not shown in FIG. 2) for clamping the bone anchors 20 both linearly and polyaxially relative to the plate 30.

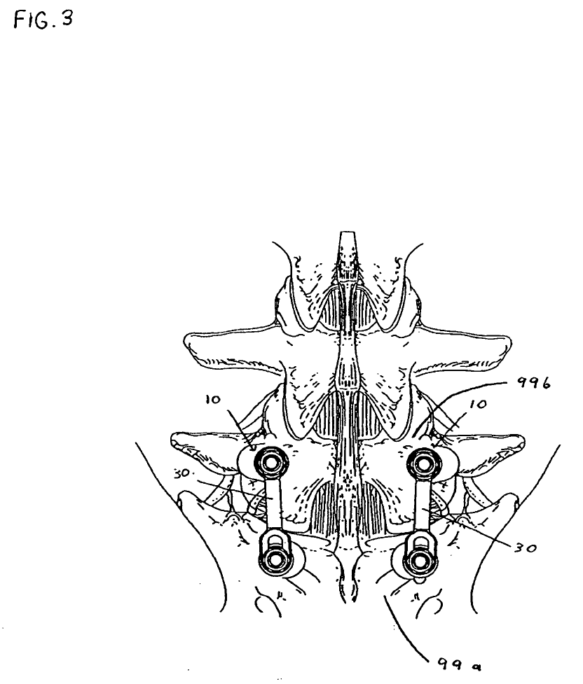

In general use, the fixation device 10 is anchored to bones such as vertebral bodies 99a, 99b (shown in FIG. 3) desired to be stabilized. The fixation device 10 can be anchored to the vertebral bodies 99a, 99b by threading the bone anchors 20 into the vertebral bodies 99a, 99b. Torque for driving the anchors 20 can be provided by a tool (not shown) such as a wrench or other surgical tool. After threading the anchors 20 into the vertebral bodies 99a, 99b, the vertebral bodies 99a, 99b can be distracted apart, compressed together or otherwise moved to a desired relative positioning. The plate 30 can then be placed over the anchors 20 with the toggle bolts 50 received through the fastener openings 40 of the plate and the spherical heads 24 of the anchors 20 fitting within the cup-shaped washers 60. The washers 60 can slide along the plate openings 40 to facilitate placement of the plate 30 over the bone anchors 20. The polyaxial configuration of the bone anchors 20 allows the plate 30 pivot relative to the bone anchors 20. Once the plate 30 is placed over the bone anchors 20, the nuts 90 are threaded onto the toggle bolts 50 clamping the anchors to the plate. The anchors 20 are preferably clamped with sufficient force to prevent the spherical heads 24 from pivoting relative to the plate 30 and to prevent the washers 60 from sliding relative to the plate 30. In this manner, the fixation device 10 forms a stabilizing construct or framework that braces the vertebral bodies 99a, 99b to maintain the desired spacial relationship between the vertebral bodies 99a, 99b.

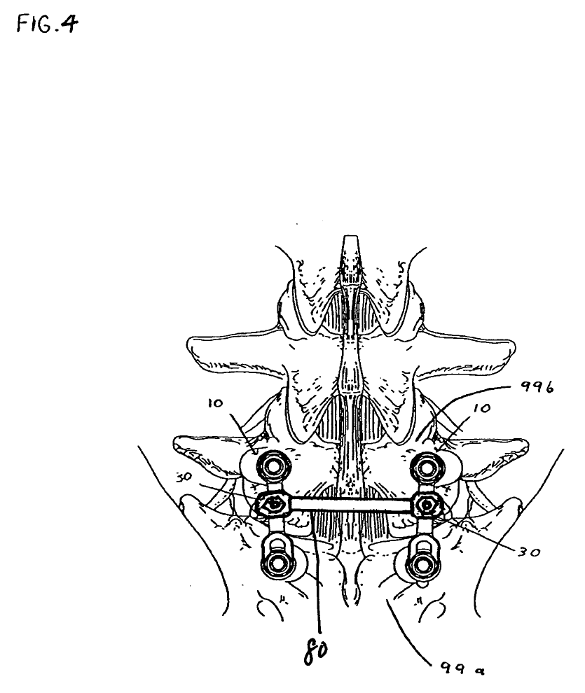

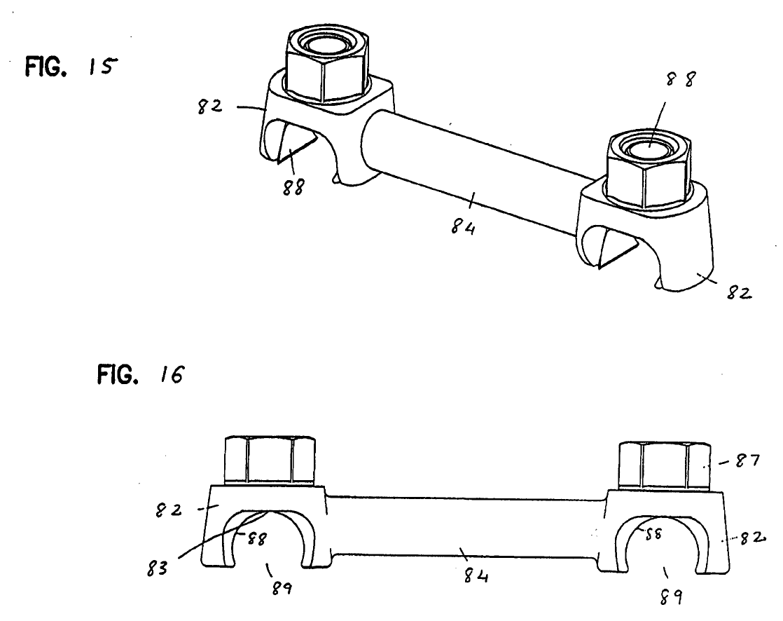

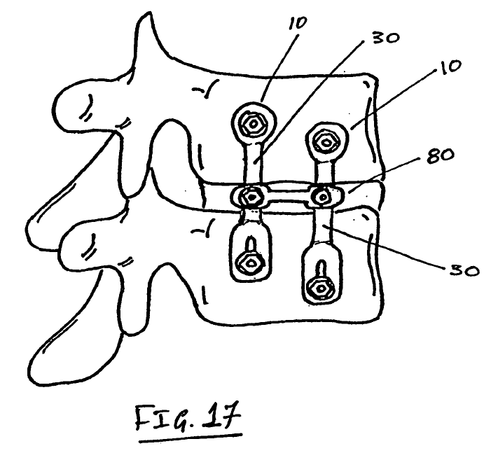

In FIG. 3, the fixation device 10 is shown as being mounted on the human spine from a posterior approach. As shown in FIG. 4, if desired, a transverse connector 80, such as the one shown in FIGS. 15 and 16, interconnecting two plates 30 transversely, in a direction generally perpendicular to the spine, can also be utilized in posterior applications.

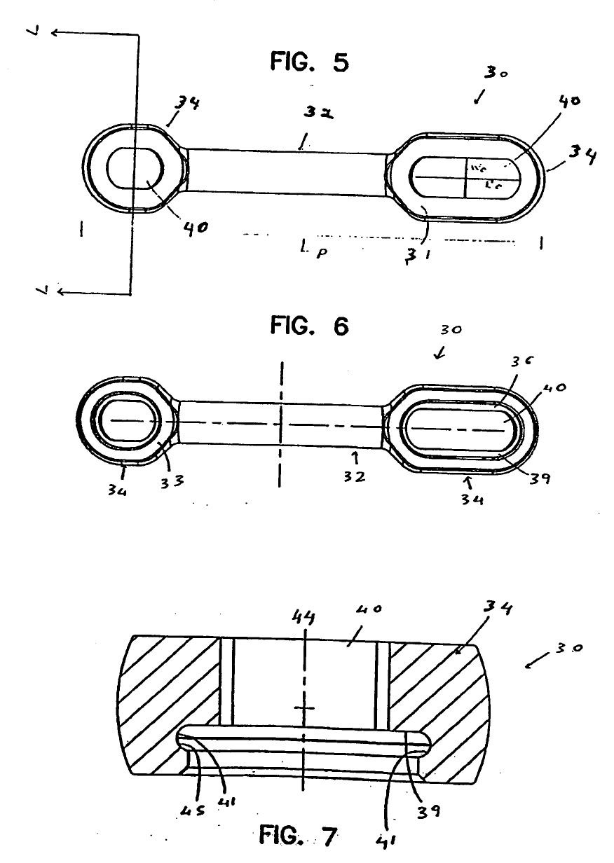

Referring to FIGS. 5-7, the plate 30 of the fixation device 10 includes a top surface 31, a bottom surface 33, and a length Lp. Along the length Lp, the plate 30 includes receiver portions 34 connected by bridge portions 32. The receiver portions 34 are configured to define the fastener openings 40. In certain embodiments, the receiver portions 34 can have generally rectangular transverse cross-sections such that the top and bottom surfaces 31, 33 are generally planar and parallel at the receiver portions 34 (see FIG. 7).

The plate 30 may include any number of receiver portions 34 along its length Lp, with each receiver portion 34 defining one or more fastener openings 40. In FIGS. 1-7, the plate 30 is depicted with one fastener opening 40 for each bone anchor 20 that is coupled to the plate 30. In other embodiments, the plate may instead include one large fastener opening that can accommodate at least two bone anchors 20 coupled to the plate.

The fastener openings 40 are generally depicted as elongate elliptical slots. The lengths of the slots can vary from opening to opening to provide varying degrees of adjustability. In certain embodiments, the lengths of the slots can be the same. In other embodiments, only one of the slots may be configured to allow adjustment between the anchors and the plate. In other embodiments, the fastener openings can be of other shapes such as a rectangle, a circle, a square, and etc. In certain preferred embodiments, in order to minimize the sizes of the components of the fixation device, the receiver portions 34 of the plate may be shaped to match the fastener openings 40 defined within the receiver portions 34. In other certain embodiments, the receiver portions may have different shapes than the fastener openings. Each fastener opening 40 includes an opening length Lₒ and an opening width Wₒ. Each fastener opening 40 also includes a longitudinal axis 44, as seen in FIG. 7.

As noted before, in the embodiments of the orthopedic fixation device, wherein the plate includes more than one receiver portion, the plate includes bridge portion(s) connecting each of the receiver portions. A bridge portion 32 of the plate 30 is illustrated in FIGS. 1-6 with a generally circular cross-section that transitions into the shape of the receiver portions 34. In other embodiments, the bridge portions may have cross-sectional shapes such as a square, a rectangle, a triangle or any polygon.

As seen in the bottom view of the plate 30 in FIG. 6, the receiver portions 34 of the plate 30 define a track 36 surrounding the perimeter of the fastener opening 40. The track 36 provides a path for the washer 60 to linearly slide along the length LQ of opening 40. The track 36 includes a track surface 39 on which the washer 60 slides along. The track surface 39, as seen in FIG. 7, may have portions 41 that extend into he material of the plate 30. The extended portions 41 essentially define a side groove 45 for the washer to slide along. As will be later discussed in more detail, the side groove 45 is adapted to prevent detachment for those embodiments of slidable washers that include top flange portions.

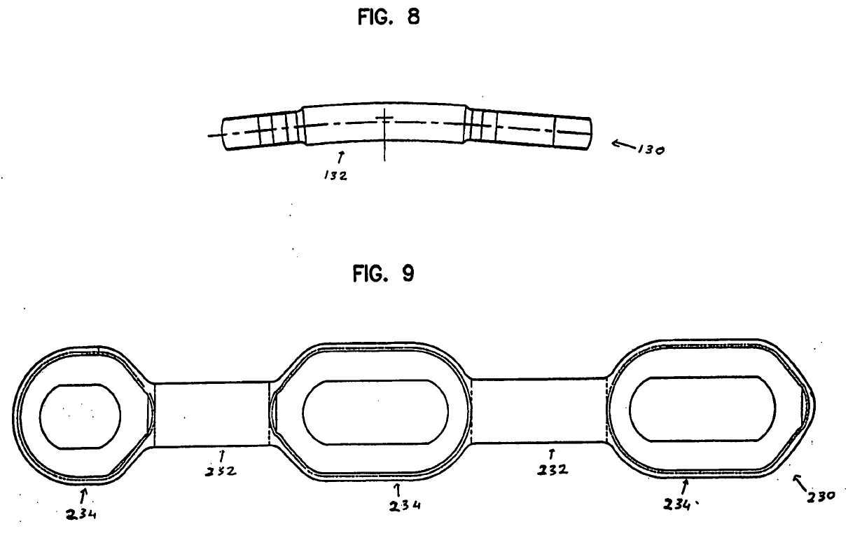

FIG. 8 illustrates another embodiment of a plate 130. The plate 130 includes a bridge portion 132 that is bent to match the contour of the spine to accommodate patient anatomy. It will be understood that the bridge portions can be bent in any direction to accommodate patient anatomy.

FIG. 9 illustrates another embodiment of a plate 230 including three receiver portions 234 and two bridge portions 232. As discussed previously, the plate of the spinal fixation device may include any number of receiver portions and bridge portions.

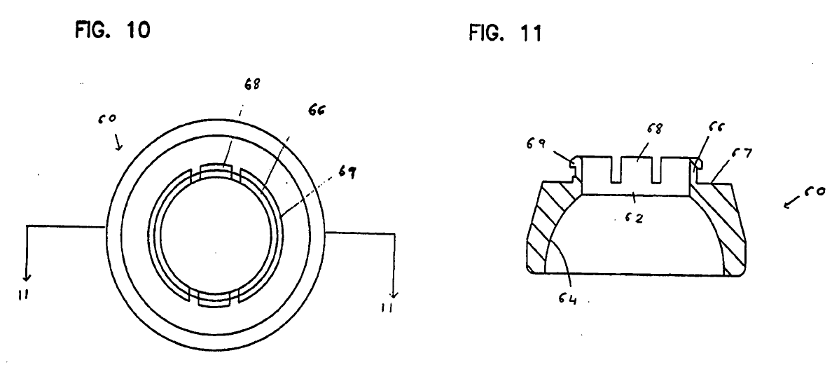

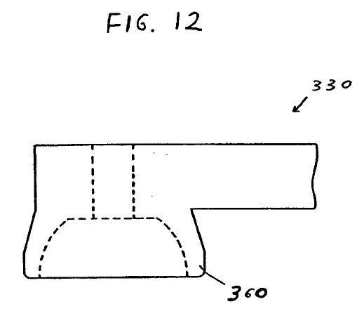

The cup-shaped washer 60 of the fixation device 10 is illustrated in FIGS. 10 and 11. FIG. 10 illustrates a top view of the washer 60 and FIG. 11 illustrates a cross-sectional view of the washer 60 of FIG. 10 taken along line 11-11 of FIG. 10.

The washer 60 is mounted between the plate 30 and the bone anchor 20 and provides for linear adjustability of the fixation device 10. The washer 60 generally includes a cup-shaped interior surface 64 shaped to fit over the spherical head 24 of the bone anchor 20 to allow for polyaxial movement of the bone anchor 20 within the washer 60. Although the exterior surface of the washer 60 can be of various shapes, it is preferably shaped to match the interior surface to minimize component sizes.

The washer 60 includes a top surface 67 and an extended portion 66 protruding upwardly from the top surface 67. The extended portion 66 of the washer 60 is adapted to allow the washer to slide along the track 36 of the plate 30 while the top surface 67 is adapted to abut and slide along the bottom surface 33 of the plate 30.

As depicted in FIGS. 10 and 11, the extended portion 66 of the washer 60 may include a flange 69 extending out radially from the extended portion 66. The flange 69 is adapted to be captured within and slide along the side groove 45 of the track 36.

The extended portions 66 may include arms 68 adapted to elastically move radially inwardly and then outwardly to enable the flange 69 to fit into the side groove 45.

The washer may also include an extended portion without a flange. In such an embodiment, the extended portion is sized such that it abuts and slides along the track surface 39 while the top surface 67 abuts the bottom surface of the plate. In such an embodiment of the washer, the extended portion is not trapped within the side groove 45 and is disengageable until final clamping of the device occurs.

The washer 60 is linearly slidably coupled to the plate 30 in such a way that the washer 60 can be tightened at any point along the track 36 along the length Lₒ of the fastener opening 40. Thus, the washer 60 and the plate 30 include an infinite number of points of linear adjustment relative to each other along the entire length Lₒ of the opening 40.

In other embodiments of the fixation device, there may be structures along the track 36 (e.g., notches, depressions, tabs, etc.) that limit the relative linear adjustment of the washer 60 and the plate 30 to discrete points along the length Lₒ of the opening 40.

The washer 60 includes a through-hole 62 that communicates with the fastener opening 40 of the plate 30 as the washer 60 slides along the track 36. The bolt end 54 of the toggle bolt 50 is inserted through the through hole 62 and fastened to the plate 30 by the nut 90.

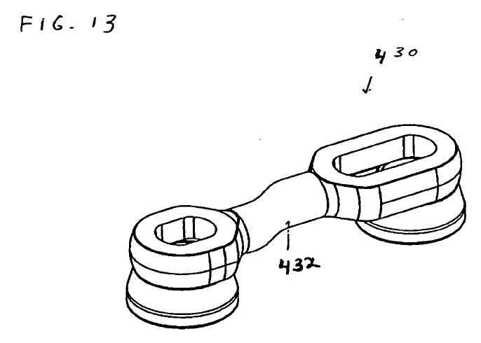

FIG. 12 illustrates a partial side view of another embodiment of a plate 330, wherein the plate 330 includes an integral, non-slidable washer 360. The integral washer 360 allows for polyaxial adjustment of the bone anchor 20 relative to the plate 330 without allowing for linear adjustment between the two components. The plate of the fixation device may include one or more such integral non-slidable washers. In certain embodiments, the one or more adjustable washers can be used at other positions along the length of the plate 330.

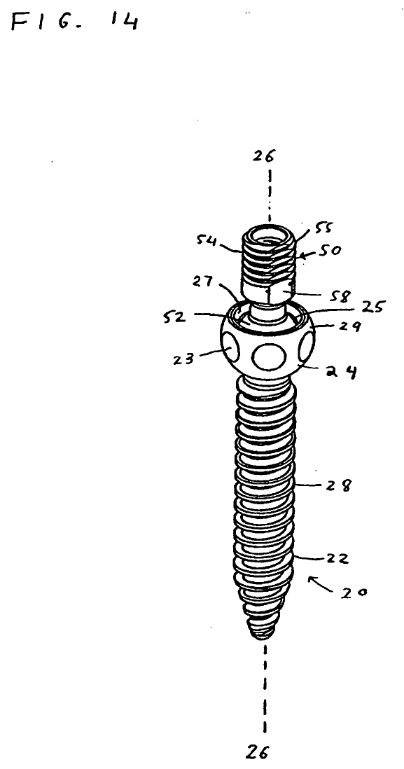

In FIG. 13, a perspective view of another embodiment of a plate 430 of the fixation system is illustrated, with the washers 60 of FIGS. 1-4 and 10-11 mounted thereon. The plate 430 includes a stepped bridge portion 432. A stepped, two-tiered bridge portion 432, such as the one included on plate 430, may be used to accommodate bony structures that may be located in between the bone anchors.

FIG. 14 illustrates the bone anchor 20 of the orthopedic fixation device 10. The bone anchor 20 is shown coupled to the toggle bolt 50 of the fixation device 10. In FIG. 14, the bone anchor 20 is depicted as a pedicle screw. The bone anchor can also include structures such as pins, hooks, expandable anchors, barbed anchors or other structures. The bone anchor 20 includes a bone engaging end 22, a generally spherical head 24, and a longitudinal axis 26 running therethrough. The bone-engaging end 22 preferably includes external threads 28 for screwing the bone anchor 20 into bone material. The spherical head 24 is shaped to allow for polyaxial movement of the bone anchor 20 before final clamping. The spherical head 24 includes an exterior surface 29 and an interior surface 27. As shown in FIG. 14, the exterior surface 29 of the anchor 20 may include structures 23, e.g., flat walls, for driving the anchor 20 into bone via use of a surgical tool (not shown). The spherical head 24 of the bone anchor 20 is sized and contoured to fit within the cup-shaped washer 60. The exterior surface 29 of the spherical head 24 is adapted to slide against the interior surface 64 of the washer 60 giving the bone anchor 20 a range of motion throughout a 360-degree pattern from the longitudinal axis 44 of the fastener opening 40.

The interior surface 27 of the head 24 defines an internal cavity, a socket 25, adapted to receive a ball end 52 portion of the toggle bolt 50. The internal cavity 25 preferably has a generally spherical shape to form a ball/socket configuration with the ball end 52 of the toggle bolt 50. This ball/socket configuration gives the bone anchor 20 a polyaxial freedom of movement relative to the toggle bolt 50.

A retainer 70, best illustrated in FIG. 2, is used to secure the ball end 52 of the toggle bolt 50 within the socket 25 of the anchor 20. The retainer 70, as depicted in FIG. 2, is essentially a sleeve of a generally cylindrical shape with an interior surface 72 and an exterior surface 74. The exterior surface 74 of the retainer 70 is shaped to contour to the internal surface 27 of the spherical head 24. The retainer 70 is inserted within the socket 25 of the spherical head 24 after the ball end 52 of the toggle bolt 50 is received within the socket 25. The retainer 70, once engaged within the socket 25 surrounding the ball end 52, prevents the ball end 52 from exiting the socket 25 of the spherical head 24. As seen in FIG. 2, the interior surface 72 of the retainer 70 tapers inwardly from the bottom to the top of the retainer forming a top rim 73. The top rim 73 of the retainer 70 is sized to be smaller than the diameter of the ball end 52 of the toggle bolt 50 to prevent the toggle bolt 50 from exiting the socket 25.

The interior surface 72 of the retainer 70 is contoured to provide a snug but smooth fit with the ball end 52 of the toggle bolt 50 allowing for slidable polyaxial movement of the ball end 52 within the socket 25.

The retainer 70 can be coupled to the interior surface 27 of the spherical head 24 in a number of ways including welding, threading, snap fitting, and etc. Accordingly, the interior surface 27 of the spherical head 24 and the exterior surface 74 of the retainer may include intermating parts depending on the coupling method used. Such parts may include structures such as ramps, tabs, internal and external threads or etc. FIG. 2 illustrates a retainer 70 that has been welded to the spherical head 24 of the bone anchor 20.

The toggle bolt 50 of the orthopedic fixation device 10 is shown in FIG. 14 along with the bone anchor 20. The toggle bolt 50, as discussed above, includes a ball end 52 and a connected bolt end 54. The bolt end 54 is sized to fit through the through hole 62 of the washer 60 and the fastener opening 40 of the plate 30. As depicted in FIG. 14, the bolt end 54 may include external threads 55 for engaging a nut 90. It will be understood that other structures are also possible for clamping the toggle bolt 50 to the plate 30.

Once the bolt end 54 of the toggle bolt 50 is inserted through the through hole 62 of the washer 60 and the fastener opening 40 of the plate 30, the nut 90 is fastened onto the threads 55 of the bolt end 54. In this manner, the spherical head 24 of the bone anchor 20 is clamped against the inside-of the washer 60 to resist polyaxial movement and the top of the washer 60 is damped against the underside of the plate 30 to resist linear movement.

The bolt end 54 of the toggle bolt 50 may include structure for countering the torque used in threading of the nut 90. For example, in FIG. 14, the bolt end 54 of the toggle bolt is depicted as having a non-circular cross-sectional shape. The bolt end of the toggle bolt includes generally flat surfaces 58 such that, once the bolt end 54 is inserted within the opening 40 of the plate 30, the toggle bolt 50 cannot rotate relative to the plate.

In FIGS. 15 and 16, an embodiment of a transverse connector 80 that may be utilized with the various orthopedic fixation devices illustrated in FIGS. 1-14 is shown. FIG. 15 illustrates a perspective view of the transverse connector 80 and FIG. 16 illustrates a side view of the transverse connector 80.

The transverse connector 80 is used to interconnect at least two plates 30. The transverse connector 80 includes at least two plate engagement portions 82 separated by an intermediate portion 84. Although depicted as a circle, the intermediate portion 84 may include a cross-sectional shape of any polygon. Each plate engagement portion 82 defines a slot 89 for receiving the bridge portions of the plates of the fixation device. The slot 89 can be of various shapes and sizes depending on the bridge portions of the plates that are interconnected.

Each plate engagement portion 82 also includes a bore 83. A clamping bolt 88 is inserted through the bore 83 and engaged by a nut 87. As the nut 87 is turned about the exterior threads of the clamping bolt 88, the bridge portion 32 of the plate 30 is clamped within the slot 89 of the transverse connector 80. In other embodiments, locking arrangements other than bolt/nut arrangements can be used to provide tightening of the plate 30 to the transverse connector 80. The transverse connector may include more than two plate engagement portions 82 to interconnect more than two plates 30.

FIG. 17 illustrates the spinal fixation device 10 mounted to a lateral side of the vertebral bodies. While two devices 10 are mounted in line with the axis of the spine in this illustrative embodiment, fewer or more devices 10 can be used. If desired, a transverse connector 80, such as the one shown in FIGS. 15 and 16, interconnecting two plates 30 transversely, in a direction generally perpendicular to the spine, can also be utilized. In addition to posterior and lateral placement discussed above, the devices and associated components can be installed in any other suitable portions of the vertebrae, including anterior and anterior-lateral portions. A variety of directions of surgical approaches well known in the art can be used.

The various components of the devices disclosed herein (e.g., the washers, the plates, the bone anchors, the toggle bolts, the retainers, and the transverse connectors) can be made of any number of different types of biocompatible materials. Example materials include materials such as Titanium, Nitinol, Stainless Steel, and other materials.

From the foregoing detailed description it will be evident that modifications and variations can be made in the devices of the invention without departing from the spirit or the scope of the invention. Therefore, it is intended that all modifications and variations not departing from the spirit of the invention come within the scope of the claims and their equivalents.

We claim:

1. An orthopedic fixation device comprising:
an elongated plate including at least two fastener openings;
at least two cup-shaped washers;
anchors including fastening portions that extend through the washers and the fastener openings;
the plate defining at least one elongated track that extends along at least one of the fastener openings;
at least one of the cup-shaped washers being configured to slide along the track.

2. An orthopedic fixation device according to claim 1, wherein the anchor includes a generally spherical head for polyaxial movement of the anchor relative to the washer.

3. An orthopedic fixation device according to claim 2, wherein the fastening portion of the anchor includes a toggle bolt with a bolt end and a ball end, wherein the ball end is coupled to a ball socket defined within the head of the anchor.

4. An orthopedic fixation device according to claim 3, further comprising a nut adapted to interlock with the bolt end of the toggle bolt to clamp the anchor to the plate.

5. An orthopedic fixation device according to claim 3, wherein the head of the anchor includes a polyaxial freedom of movement relative to the toggle bolt prior to being clamped to the plate.

6. An orthopedic fixation device according to claim 4, wherein the anchor includes a polyaxial freedom of movement relative to the plate, the washer, and the nut.

7. An orthopedic fixation device according to claim 1, wherein at least one of the cup-shaped washers includes a top flange that is captured within the track that allows the washer to slide.

8. An orthopedic fixation device according to claim 1, wherein each of the fastener openings includes a length and wherein at least one of the cup-shaped washers and the plate include an infinite number of points of linear adjustment relative to each other along the entire length of at least one of the fastener openings.

9. An orthopedic fixation device according to claim 1, wherein the plate includes a bent portion.

10. An orthopedic fixation device according to claim 1, wherein the plate includes a stepped portion.

11. An orthopedic fixation device according to claim 1, wherein the plate includes an intermediate portion between the fastener openings, the intermediate portion having a rounded cross section.

12. An orthopedic fixation device comprising:
a plate defining a length, the plate including first and second receiver portions linked by a bridge portion, the first receiver portion defining a first opening that is elongated in a direction that extends along the length of the plate and the second receiver portion defining a second opening that is elongated in a direction that extends along the length of the plate, the first and the second openings also extending through the plate from a bottom side of the plate to a top side of the plate;
the bottom side of the plate including first and second tracks that extend respectively along the first and second openings; and
cup-shaped washers mounted to slide along the tracks.

13. An orthopedic fixation device according to claim 12, wherein the cup-shaped washers include top flanges that are captured within the tracks that allow the washers to slide.

14. An orthopedic fixation device according to claim 12, wherein the bridge portion includes a round cross-section and the plate includes flat top and bottom surfaces at first and second receiver portions.

15. An orthopedic fixation device according to claim 12 further comprising anchors including fastening portions that extend through the washers and the openings.

16. An orthopedic fixation device according to claim 15, wherein the fastening portions of the anchors include toggle bolts with bolt ends and ball ends, wherein the ball ends are coupled to ball sockets defined within heads of the anchors.

17. An orthopedic fixation device comprising:
an elongated plate including a first end, a second end, a top side, and a bottom side, the plate including an integral cup-shaped washer;
wherein the integral cup-shaped washer projects downwardly from the bottom side of the plate.

18. An orthopedic fixation device according to claim 17, wherein the plate includes first and second fastener openings.

19. An orthopedic fixation device according to claim 18, wherein the plate includes first, second, and third fastener openings.

20. An orthopedic fixation device according to claim 18, wherein the first and second fastener openings are defined respectively by first and second receiver portions having flat top and bottom surfaces, the receiver portions linked by a bridge portion having a round cross-section.

21. An orthopedic fixation device according to claim 18, wherein the fastener openings include slots elongated in a direction that extends along a length of the plate.

22. An orthopedic fixation device according to claim 21, wherein one of the slots has a different length than another one of the slots.

23. A method of fixing the spine comprising the steps of:
securing an anchor to the spine;
positioning an elongated plate in a direction generally parallel to the spine;
sliding at least one washer relative to the plate in a direction generally parallel to the spine; and
securing the plate to the spine by inserting a fastening portion of the anchor through the washer and the plate.

24. A method of fixing the spine according to claim 23, further comprising mounting the elongated plate to the spine from a posterior approach.

25. A method of fixing the spine according to claim 23, further comprising mounting the elongated plate to the spine from a lateral approach.

26. An orthopedic fixation device comprising:
a plate defining a fastener opening including a length, the plate connecting at least two bone anchors;
a fastener adapted to clamp each anchor to the plate; and
at least one cup-shaped washer linearly slidably coupled to the plate, slidable along the length of the fastener opening, coupled in such a way that the washer and the plate include an infinite number of points of linear adjustment relative to each other along the entire length of the fastener opening, wherein each anchor includes a generally spherical head shaped to fit inside the cup-shaped washer, the head allowing for polyaxial freedom of movement;
wherein each anchor includes a polyaxial freedom of movement relative to the fastener prior to being clamped to the plate.

27. An orthopedic fixation system comprising:
at least two orthopedic fixation devices, each orthopedic fixation device including:
an elongated plate including at least one fastener opening
at least two cup-shaped washers mounted to the plate; and
an anchor mounted to each washer;
wherein each cup-shaped washer slides on a track that extends along each fastener opening and wherein each cup-shaped washer includes a top flange that is captured within the track that allows it to slide, and
a transverse connector for interconnecting two elongated plates.

28. An orthopedic fixation system according to claim 27, wherein the transverse connector is coupled to the plate at a connection point between the anchors.

29. An orthopedic fixation system according to claim 28, wherein the plate includes a rounded cross-section at the connection point.

30. An orthopedic fixation system comprising at least two orthopedic fixation devices and at least one connector for interconnecting the two orthopedic fixation devices, each of said connector and each of said orthopedic fixation device comprising
an elongated member having a first end and a second end, a first fastener opening at said first end of said elongated member, a second fastener opening at said second end of said elongated member, said elongated member defining at least one elongated track extending along at least one of the fastener openings;
at least two cup-shaped washers wherein at least one of said cup-shaped washer is configured to slide along said elongated track; and
anchors including fastening portions that extend through said washers and said first and second fastener openings.

31. The orthopedic fixation system of claim 30 wherein said connector is configured to provide a transverse interconnect said orthopedic fixation devices when the fixation devices are attached to bone portions in a side-by-side fashion.

32. The orthopedic fixation system of claim 30 wherein each anchor is configured for polyaxial movement relative to the washer.

33. The orthopedic fixation system of claim 32 wherein the fastening portion of each anchor includes a toggle bolt with a bolt end and a ball end, wherein said ball end is coupled to a ball socket defined within the head of said anchor.

34. The orthopedic fixation system of claim 33 further comprising a nut adapted to interlock with said bolt end of said toggle bolt to clamp said anchor to said elongated member.

35. The orthopedic fixation system of claim 34 wherein said anchor includes a polyaxial freedom of movement relative to said elongated member, said washer, and said nut.

36. The orthopedic fixation system of claim 33 wherein the head of said anchor includes a polyaxial freedom of movement relative to said toggle bolt prior to being clamped to said elongated member.

37. The orthopedic fixation system of claim 30 wherein at least one of said fastener opening includes a length, and wherein at least one of said cup-shaped washer and said elongated member include an infinite number of points for linear adjustment relative to each other along the entire length of said fastener opening.

38. A method for fixing the spine comprising securing an orthopedic fixation device to lateral portions of two adjacent vertebrae, the device comprising
an elongated plate including at least two fastener openings;
at least two cup-shaped washers;
anchors including fastening portions that extend through the washers and the fastener openings;
the plate defining at least one elongated track that extends along at least one of the fastener openings; and
at least one of the cup-shaped washers being configured to slide along the track.

39. The method of claim 38 further comprising securing an additional orthopedic fixation device to lateral portions of said adjacent vertebrae, the additional device comprising
an elongated plate including at least two fastener openings;
at least two cup-shaped washers;
anchors including fastening portions that extend through the washers and the fastener openings;
the plate defining at least one elongated track that extends along at least one of the fastener openings;
at least one of the cup-shaped washers being configured to slide along the track; and
interconnecting the devices using a connector.

40. The method for fixing the spine according to claim 39 wherein each of the securing steps comprises securing each of said orthopedic fixation devices in a direction generally parallel to the spine.

41. The method for fixing the spine according to claim 39 comprising the step of securing said first and second orthopedic fixation devices to the spine from a lateral approach.

42. The method for fixing the spine according to claim 39 comprising the step of positioning said connector in a direction generally perpendicular to the spine.

43. An orthopedic fixation system comprising
a first means for securing a pair of adjacent vertebrae;
a second means for securing said pair of adjacent vertebrae; and
a third means for interconnecting together said first and second means.

44. The orthopedic fixation system of claim 43 wherein said pair of adjacent vertebrae are secured in a generally lateral direction.

45. The orthopedic fixation system of claim 43 wherein said third means interconnects said first and second means in a generally transverse direction.

46. The orthopedic fixation system of claim 43 wherein said first means is in a direction generally parallel to the spine.

47. The orthopedic fixation system of claim 43 wherein said second means is in a direction generally parallel to the spine.

48. The orthopedic fixation system of claim 43 wherein said third means is in a direction generally perpendicular to the spine.
